(19) United States
(12) Patent Application Publication
(10) Pub. No.: US 2026/0020846 A1
(43) Pub. Date: Jan. 22, 2026

(54) MINIATURE BIOPSY DEVICE

(71) Applicant: BIONAUT LABS LTD., Herzliya (IL)

(72) Inventors: Jason SHIH, Pasadena, CA (US); Florent CROS, Los Angeles, CA (US); Alex KISELYOV, San Diego, CA (US)

(73) Assignee: BIONAUT LABS LTD., Herzliya (IL)

(21) Appl. No.: 19/270,254

(22) Filed: Jul. 15, 2025

Related U.S. Application Data
(60) Provisional application No. 63/674,226, filed on Jul. 22, 2024.

(51) Int. Cl. A61B 10/02; A61B 10/00

(52) U.S. Cl. CPC A61B 10/0275; A61B 10/0096

(57) ABSTRACT

Provided is a miniature biopsy device for use in a patient. The miniature biopsy device is configured to be operated remotely. store and collect a biopsy sample within the miniature device. Also provided is the related system configured to carry out such an operation. and the related methods for use in biopsies.

### CROSS REFERENCE TO RELATED APPLICATIONS

This application claims the benefit of priority of U.S. Provisional Application No. 63/674,226, filed Jul. 22, 2024, hereby incorporated by reference.

### FIELD OF THE INVENTION

The presently disclosed subject matter relates to miniature devices for use in biopsies. The device is steered remotely to a site within a patient, performs a biopsy and stores the biopsy sample for retrieval. Related systems and methods are disclosed.

### BACKGROUND

A biopsy is a medical procedure involving the removal of a sample of tissue or cells from the body for examination under a microscope. It is crucial for diagnosing various medical conditions, including cancers and infections. Over time, advancements in imaging techniques and minimally invasive procedures have refined biopsy processes, reducing patient discomfort and recovery times. Challenges include ensuring accurate sampling representative of the lesion, minimizing risks such as bleeding or infection, and interpreting complex results to guide appropriate treatment decisions.

Performing biopsies can be distressing to the patient and many uncertainties arise from the external probing of surgical instruments to reach a target area. Current biopsy methods typically involve externally-inserted surgical tools such as needles, probes, and endoscopes to a target location. An aim of the present invention is to provide a miniature device that maneuvers inside a patient, collects a biopsy sample and stores it for retrieval and examination. Being able to collect a biopsy sample with minimal risks, in a self-contained remotely-controlled miniature device provides a significant advantage over current biopsy techniques.

### SUMMARY

In one embodiment the invention provides a miniature device for use in a system configured to store a biopsy sample collected at a biopsy site in a patient, the miniature device comprising:
at least one steering portion comprising a magnetic material; and
a window comprised in the at least one steering portion through which the biopsy sample passes into at least one chamber within the miniature device.

In one embodiment the miniature device further comprises a surgical element comprising at least one sharp edge and configured to remove the biopsy sample for storage within the miniature device.

In one embodiment the at least one sharp edge is selected from: smooth edge, serrated edge, hollow grind, beveled edge, and scalloped edge.

In one embodiment the surgical element is a cylinder comprising a longitudinal opening with the at least one sharp edge, the surgical element being configured to rotate within the miniature device along its longitudinal axis to cut the biopsy sample as the at least one sharp edge passes across the window.

In one embodiment the miniature device is further configured to seal the at least one chamber when the longitudinal opening is positioned away from the window.

In one embodiment the surgical element is a cylinder comprising the at least one sharp edge, the surgical element being configured to move backwards to open the window and forwards to close the window along the longitudinal axis of the miniature device, wherein the sharp edge cuts the biopsy sample for storage into the at least one chamber as the sharp edge moves forwards to close the window.

In one embodiment the surgical element is a cylinder comprising the at least one sharp edge, the surgical element being configured to move backwards to extend out of the window at the back end of the miniature device and forwards to retract into the miniature device along the longitudinal axis of the miniature device, wherein the at least one sharp edge cuts the biopsy sample when extending out of the back end of the miniature device for storage into the at least one chamber when the surgical element retracts into the miniature device.

In one embodiment the surgical element comprises the at least one sharp edge configured at least partially at the edges of the window at the back end of the miniature device and wherein the biopsy sample is cut when the miniature device moves backwards into the biopsy site.

In one embodiment the window further comprises a hinged door comprising at least one sharp edge, the at least one sharp edge being positioned such that the biopsy sample is cut and directed into the at least one chamber for storage when the miniature device moves forwards.

In one embodiment the hinged door is configured to close, sealing the biopsy sample inside the at least one chamber.

In one embodiment the miniature device further comprises a pocket door positioned at the side of the window wherein the surgical element is configured to retract into the body of the miniature device via the pocket door to open the window and extend out of the body of the miniature device via the pocket door to close the window thereby cutting the biopsy sample for storage within the at least one chamber.

In one embodiment the miniature device further comprises a tapered end.

In one embodiment the steering portion comprises a non-magnetic shell at least partially surrounding the magnetic material.

In one embodiment the invention provides a system configured to remove and store a biopsy sample at a biopsy site in a patient, the system comprising at least one miniature device according to any one of the preceding claims, the system further comprising a magnetic inducing apparatus configured to be operated to generate a varying magnetic field, thereby remotely controlling motion of the miniature device.

In one embodiment the invention provides a biopsy method comprising:
inserting the miniature device of the invention into the patient via an entry point;
remotely controlling the motion of the miniature device towards the biopsy site;
removing the biopsy sample from the biopsy site for storage in the at least one chamber; and
retrieving the miniature device via an exit point.

In one embodiment the inserting is carried out by any of the following selected from: needle, catheter, or a combination thereof.

In one embodiment the motion is carried out in a linear and/or non-linear trajectory through the patient.

In one embodiment the miniature device passes through any of the following selected from: tissue, channels, tracts, ducts, cavities, epidural space, vessels, organs, potential space, interfascial planes, interorgan space, or a combination thereof.

In one embodiment the channels are pre-made channels, naturally occurring channels, or a combination thereof.

In one embodiment the window is open when at the biopsy site and closed when not at the biopsy site.

In one embodiment the removing is carried out by moving the miniature device relative to the biopsy site, moving the surgical element relative to the biopsy site, or a combination thereof, whilst is contact with the biopsy site, thereby removing the biopsy sample.

In one embodiment the moving the miniature device relative to the biopsy site, moving of the surgical element relative to the biopsy site, or a combination thereof, is selected from: forward, backward, sideward, up, down, vibration, or a combination thereof.

In one embodiment the method further comprises closing the window of the miniature device, thereby enclosing the biopsy sample in the at least one chamber.

In one embodiment the enclosing forms a hermetic seal.

In one embodiment the entry point and the exit point are the same.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a 3D illustration of a miniature device with a closed window.

FIG. 1B it a 3D illustration of the miniature device of FIG. 1A, but with the window in the open position.

FIG. 1C is a 3D illustration of an example of a cylindrical/rotating surgical element placed in the miniature device of FIGS. 1A-1B.

FIG. 1D is a 3D illustration of another example of a rotating surgical element placed in the miniature device of FIGS. 1A-1B.

FIG. 2 shows a 3D representation of a miniature device with a surgical element (gray) which moves to open and close a window; the closing of the window both cuts the biopsy sample and stores it within the miniature device.

FIG. 3 shows an example of the miniature device wherein the surgical element is configured to extrude out of the back end of the miniature device to collect a biopsy sample and retract inwards to store the biopsy sample within.

FIG. 4 shows a 3D illustration of a miniature device with a surgical element (comprising a sharp edge) at the back of the miniature device.

FIG. 5 shows a miniature device with a surgical element as a trap door that is configured to cut a biopsy sample when in contact with, and moving past, the biopsy site.

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

The present invention provides a miniature device for biopsies. The purpose of the miniature device is generally twofold: 1) obtaining a biopsy sample, and 2) storing the biopsy sample for retrieval. In various embodiments the storage of the biopsy sample is done in a manner in which it is hermetically sealed, as will become clear. As used herein, in reference to the taking of the biopsy sample itself, the terms “taking”, “obtaining”, “collecting”, “extracting”, “removing”, “cutting”, “harvesting”, and the likes, are used interchangeably; regardless of the means by which the biopsy sample was taken. For example, the biopsy sample may be taken by surgical cutting, scraping, incision, excision, punch-biopsy, needle biopsy, etc. The terms “biopsy sample” and “sample” are understood interchangeably.

As used herein, the terms miniature device' and ‘miniature biopsy device’ are used interchangeably. The size of the device is selected as suited for a particular task. The biopsy materials can include but are not limited to: tissues, cells, fluids, or a combination thereof.

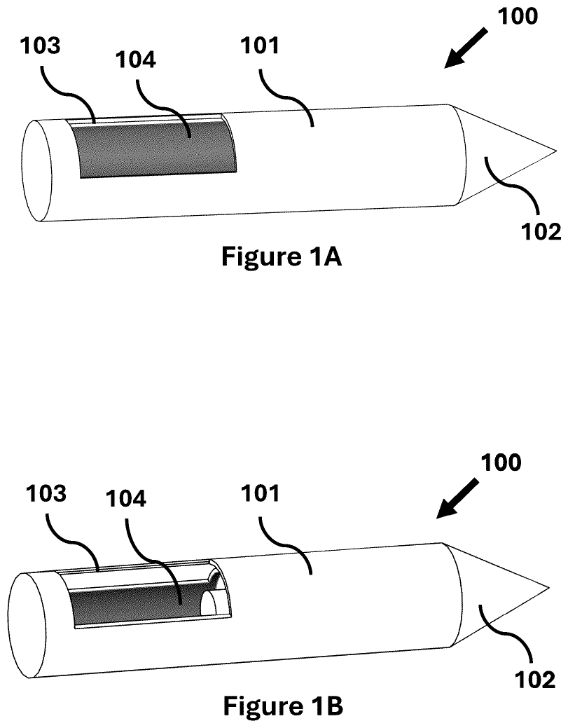

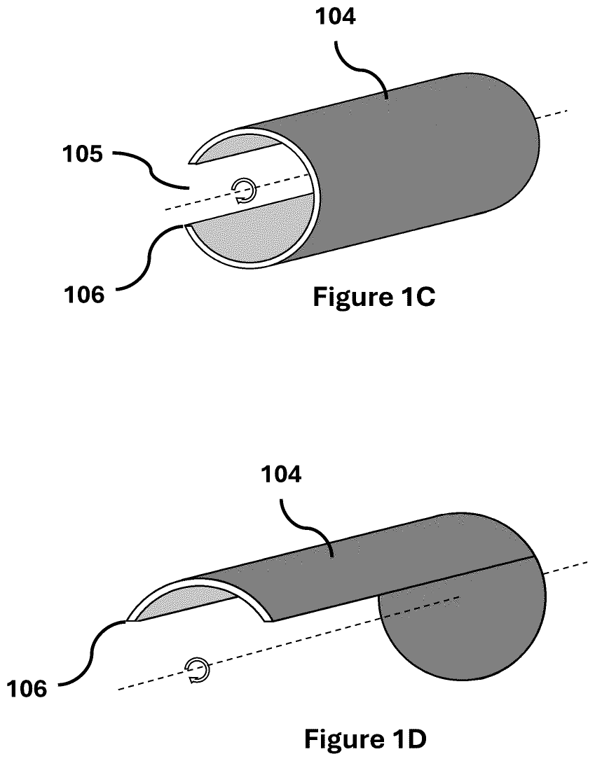

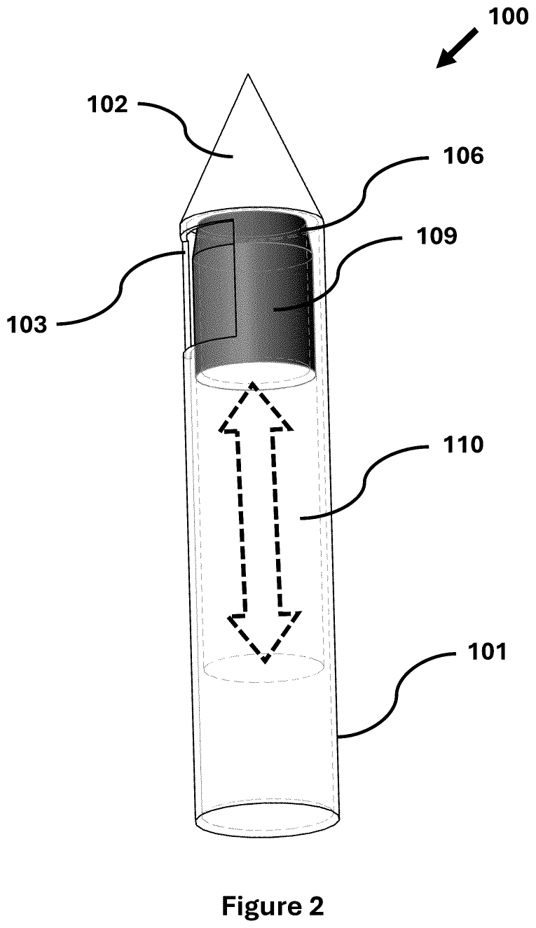

Examples will now be provided with reference to the Figures. FIGS. 1A-1D and FIG. 2 detail miniature devices where the surgical element does not extrude from the body of the miniature device. FIG. 1A shows a miniature device 100 with a body 101 which comprises at least one steering portion comprising a magnetic material. The shape of the body 101 is cylindrical and elongated. A tapered end 102 is attached to the front of the miniature device 100. The tapered end 102 can be integrated into the body 101 as a whole, or as an assembled component. The tapered end 102 provides streamlining to the miniature device 100 to assist its passage through a patient. Although the tapered end 102 depicted as a cone, the shape of the tapered end 102 can be varied and suited for purposes. For example, the tapered end 102 can be a dome shape. A window 103 is present on the body 101 of the miniature device 100 through which a biopsy sample may enter. In its simplest form, the miniature device 100 moves past a biopsy site, whilst in contact, thereby removing the biopsy sample into the miniature device 100 through the window 103. In some embodiments the window has a sharp edge (e.g., a knife-edge) on at least one part of the window frame which facilitates the cutting of the biopsy sample. In one embodiment window has at least one sharp edge. FIG. 1A shows a surgical element 104 which closes the window 103 by rotating along the longitudinal axis. The surgical element 104 being a cylindrical shape with a longitudinal gap along the length of the cylinder (also shown in FIGS. 1C and 1D). In other embodiments the window 103 comprises pocket door (not shown) into which a sliding surgical element is extended and retracted. The window 103 can be placed anywhere on the body 101 of the miniature device 100. In one embodiment the miniature device 100 comprises more than one window. In one embodiment the miniature device 100 comprises at least two windows. Each window 103 corresponds to another chamber into which a biopsy sample can be collected. The terms “chamber” and “collection chamber” (for biopsy samples) are understood interchangeably and refer to any space utilized for storage of a biopsy, or other volumetric space within the miniature device. For example, a window can be placed on the upper and lower side of the miniature device to collect two separate samples from two separate locations, each stored within their own internal chambers. In one embodiment the window comprises 1-50% of the surface of the miniature device. As defined herein, a “window” refers to any hole on the miniature device through which the biopsy sample enters (it is referred to as a ‘window’ whether it is in the open or closed configuration).

FIG. 1B shows the window 103 in the open configuration whereby the surgical element 104 has rotated to expose a gap through which a biopsy sample can now be collected. The surgical element 104 is rotated to control whether the configuration of the window 103 is in the open or the closed position. In various embodiments the window 103 remains closed until the miniature device has arrived at the biopsy site in the patient. When it arrives at the biopsy site the window 103 is opened by rotating the surgical element 104 after which the biopsy sample is collected and the window 103 is subsequently closed.

FIG. 1C shows an example of the surgical element 104 in the shape of a cylinder which has a longitudinal opening 105 along the entire length of the cylinder. The surgical element rotates on the longitudinal axis (see dotted line and arrow). In various embodiment the leading edge 106 of the longitudinal opening 105 is a sharp edge. Therefore, the surgical element 104 rotates cutting a biopsy sample that is positioned at least partially inside the miniature device through the window 103, when the longitudinal opening 105 passes across the window 103. The surgical element 104 rotates further to seal the biopsy sample inside the miniature device. In various embodiments the longitudinal opening 105 comprises part of the length of the cylinder. FIG. 1D shows another example of the surgical element 104 which rotates along the longitudinal axis (see dotted line and arrow). As shown in FIGS. 1A-1D, in order to seal the window; the surgical element needs to be at least the size of the window to ensure that the window is closed. As such, the shape of the surgical element 104 can correspond to the shape of the window 103, in various embodiments.

FIG. 2 shows a punch-biopsy type mechanism integrated into the miniature device 100. The body 101 of the miniature device 100 comprises an internal space 110 within which a cylindrical surgical element 109 comprises a sharp edge 106. The dotted arrow indicates the range of motion of the cylindrical surgical element 109 and the tapered end 102 is positioned at the front of the miniature device 100. The cylindrical surgical element 109 is retracted towards the back of the miniature device 100, opening the window 103. When the miniature device 100 is moved to the biopsy site, is in contact with biopsy material, the cylindrical surgical element 109 moves forward, closing the window 103 and cutting the biopsy sample for storage within. In various embodiments, the cylindrical surgical element 109 is configured to close by moving forwards, sealing the window 103. As understood herein, and in various embodiments, the terms “cylindrical surgical element” and “cylindrical blade” are used interchangeably.

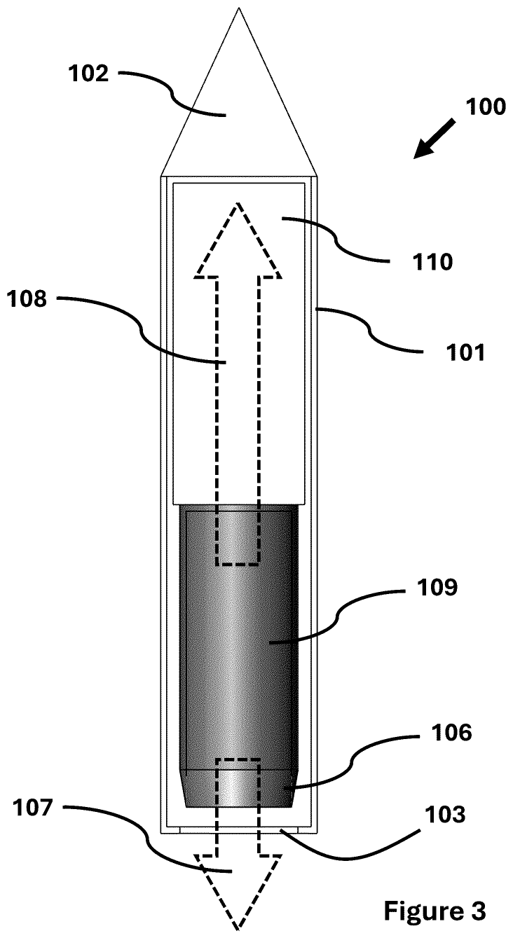

FIG. 3 shows a miniature device 100 as a punch-biopsy tool. The principle actions and operation of this example is similar to that which is shown in FIG. 2 although in FIG. 3 the window is at the back of the miniature device 100 and the cylindrical surgical element 109 extrudes outwards. The miniature device 100 comprises a body 101, tapered end 102, internal chamber 110 and a cylindrical surgical element 109 comprising a sharp edge 106 which extrudes out 107 of the back of the miniature device 100, via the window 103, to collect a biopsy sample. The cylindrical surgical element 109 then retracts into the body of the miniature device 100 to store the biopsy sample for retrieval. The window may further comprise a closing mechanism (not shown) to seal the biopsy sample inside.

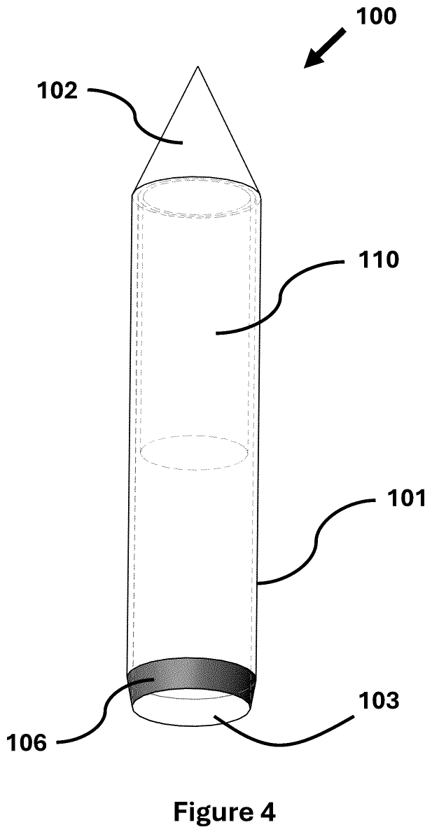

FIG. 4 shows a miniature device 100 comprising a body 101, internal chamber 100, a tapered end 102 (at the front), and a sharp edge 106 on the rim of the window 103. In this configuration the sharp edge 106 is fixed on the body 101 of the miniature device 100. A biopsy sample is collected when the miniature device 100 moves backwards into the biopsy site thereby removing the biopsy sample by means of the sharp edge 106.

In various embodiments the sharp edge 106 is selected from: smooth (straight) edge, serrated edge, hollow grind, beveled edge, and scalloped edge.

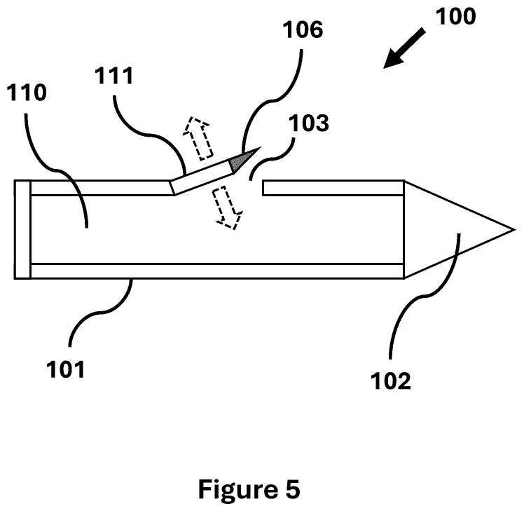

FIG. 5 shows a miniature device 100 with a hinged door 111, hinged at one edge of the window 103, the hinged door 111 further comprising a sharp edge 106 at its distal end of the hinged door. As the miniature device 100 moves past the biopsy site, whilst in contact, the biopsy sample of cut by the sharp edge 106 of the opened hinged door 111. FIG. 5 shows the sharp edge 106 as a bevel edge which is configured to direct the biopsy sample towards the interior of the miniature device 100. In this configuration, when the miniature device 100 is passing though the patient on its way to the biopsy site, the window 103 remains closed by closing the hinged door 111. At the location of the biopsy site, the hinged door 111 opens, to facilitate the cutting of the biopsy sample and its storage therein. The hinged door 111 then closes to seal the miniature device 100.

### Miniature Devices of the Invention

In one embodiment the invention provides a miniature device for use in a system configured to store a biopsy sample collected at a biopsy site in a patient, the miniature device comprising:
at least one steering portion comprising a magnetic material; and
a window comprised in the at least one steering portion through which the biopsy sample passes into at least one chamber within the miniature device.

In one embodiment the miniature device comprises at least one chamber.

In one embodiment the invention provides a miniature device comprising:
at least one steering portion comprising a magnetic material; and
a window comprised in the at least one steering portion through which the biopsy sample passes into at least one chamber within the miniature device.

In one embodiment the invention provides a miniature device for use in a system configured to store a biopsy sample collected at a biopsy site in a patient, the miniature device comprising:
a steering portion comprising a magnetic material; and
a window comprised in the at least one steering portion through which the biopsy sample passes into at least one chamber within the miniature device.

In one embodiment the invention provides a miniature device comprising:
at least one steering portion comprising a magnetic material; and
a window comprised in the at least one steering portion through which the biopsy sample passes into at least one chamber within the miniature device.

In one embodiment the invention provides a miniature device comprising:
at least one steering portion comprising a magnetic material;
at least one chamber; and
a window comprised in the at least one steering portion through which the biopsy sample passes into the at least one chamber within the miniature device.

As stated herein, the use of the miniature device serves at least two related purposes: 1) removal of biopsy sample and, 2) storage of the biopsy for retrieval. Both purposes are equally applicable separately, and in combination. Therefore, the miniature is for use to store a biopsy sample, to remove a biopsy sample, and a combination thereof. Therefore, the uses of the miniature device and their corresponding systems is not meant to limit the scope of the miniature device. Thus, the miniature devices of the invention are often referred to as being used or configured with corresponding systems to carry out at least one particular task, however, these are not limiting.

The steering portion refers to a component comprised within the miniature device that enables the motion of the miniature device by means of an external magnetic field. It is composed partially or entirely of a magnetic material. The steering portion is configured to interact with the magnetic field generated by a magnetic inducing apparatus, thereby facilitating control of the miniature device thereby e.g., remotely. In various embodiments the steering portion comprises a protective shell. More than one steering portion can be used to facilitate the motion of the miniature device. For example, the steering portion can comprise more than one magnet, whether a permanent magnet, electromagnet, or a combination thereof.

In one embodiment the miniature comprises at least one window. Where multiple windows are present in the miniature device, each window corresponds to a separate chamber comprised within the miniature device.

In one embodiment the miniature device further comprises a surgical element configured to remove the biopsy sample for storage within the miniature device. In one embodiment the surgical element comprises at least one sharp edge and is configured to remove the biopsy sample for storage within the miniature device.

In one embodiment the invention provides a miniature device comprises:
a steering portion comprising a magnetic material;
a surgical element configured to remove the biopsy sample for storage within the miniature device; and
a window comprised in the at least one steering portion through which the biopsy sample passes into at least one chamber within the miniature device.

As understood herein the “surgical element” refers to any element comprised within the miniature device that is configured to at least partially remove the biopsy sample. In one embodiment the surgical element comprises at least one sharp edge. Thus, as used herein, the surgical element and/or the sharp edge are referred to interchangeably as means to cut the biopsy sample. In one embodiment the miniature device comprises at least one surgical element. In one embodiment the surgical element is integrated into the miniature device. In one embodiment the surgical element is a cylinder comprising a longitudinal opening with at least one sharp edge, the surgical element being configured to rotate within the miniature device along its longitudinal axis to cut the biopsy sample as the at least one sharp edge passes across the window. In one embodiment the miniature device is further configured to seal the at least one chamber when the longitudinal opening is positioned away from the window.

In one embodiment the surgical element extrudes out of the miniature device to collect the biopsy sample. In one embodiment the surgical element remains within the miniature device when collecting the biological sample. When the surgical element remains within the miniature device, the biopsy sample is at least partially positioned through the window after which a motion of the surgical element within the miniature device at least partially removes the biopsy sample.

In one embodiment the surgical element is a cylinder comprising at least one sharp edge, the surgical element being configured to move backwards to open the window and forwards to close the window along the longitudinal axis of the miniature device, wherein the at least one sharp edge cuts the biopsy sample for storage into the at least one chamber as the at least one sharp edge moves forwards to close the window.

In one embodiment the surgical element is a cylinder comprising at least one sharp edge, the surgical element being configured to move backwards to extend out of the window at the back end of the miniature device and forwards to retract into the miniature device along the longitudinal axis of the miniature device, wherein the at least one sharp edge cuts the biopsy sample when extending out of the back end of the miniature device for storage into the at least one chamber when the surgical element retracts into the miniature device.

In one embodiment the surgical element comprises at least one sharp edge configured at least partially at the edges of the window at the back end of the miniature device and wherein the biopsy sample is cut when the miniature device moves backwards into the biopsy site.

In one embodiment of the miniature device the window further comprises a hinged door comprising at least one sharp edge, the at least one sharp edge being positioned such that the biopsy sample is cut and directed into the at least one chamber for storage when the miniature device moves forwards. The sharp edge of this example is positioned in a manner such that the biopsy sample is directed into the miniature device e.g., by means of a bevel shaped edge. In one embodiment the hinged door is configured to close, sealing the biopsy sample inside the at least one chamber.

As understood herein, when a compartment, chamber, or window closes, sealing at least one biopsy sample within the miniature device, this can be a hermetic seal.

In one embodiment the miniature device further comprises a pocket door positioned at the side of the window wherein the surgical element is configured to retract into the body of the miniature device via the pocket door to open the window and extend out of the body of the miniature device via the pocket door to close the window thereby cutting the biopsy sample for storage within the at least one chamber.

In one embodiment the miniature device is approximately structured as a cylinder. In one embodiment the miniature device is approximately structured as a prolate spheroid. In one embodiment the miniature device is an elongated structure. In one embodiment the miniature device further comprises a tapered end. The tapered end either being integrated into the general structure of the miniature device or as a compartment as part of an assembly of the miniature device.

In one embodiment the steering portion comprises a non-magnetic shell at least partially surrounding the magnetic material. Examples of the material comprising the non-magnetic shell include, but are not limited to: plastics, polymers, silicone rubber, metals, elastomers, ceramics, composites, and alloys.

In one embodiment the invention provides a system configured to remove and store a biopsy sample at a biopsy site in a patient, the system comprising at least one miniature device according to any one of the preceding claims, the system further comprising a magnetic inducing apparatus configured to be operated to generate a varying magnetic field, thereby remotely controlling motion of the miniature device. The system comprises at least one miniature device and a magnetic inducing apparatus configured to control the miniature device. The magnetic inducing apparatus is configured to be operated to generate a varying magnetic field and thereby remotely, i.e., from a location exterior to a patient's body, control the motion of the miniature device within the body. According to some embodiments, characteristics of the magnetic field, for example including, but not limited to, distance, directionality, intensity, gradient, time dependence/independence, etc., may be controlled by a user in order to remotely control the motion of the device.

### Biopsy Methods of the Invention

The invention provides a biopsy method. In various embodiments the methods of the invention are for use in biopsies related to neurological conditions. Examples of uses for the invention include, but are not limited to: neuro-oncology, neurology, neurodegenerative diseases, stroke as exemplified by diffuse intrinsic pontine glioma, glioblastoma, astrocytoma, meningioma, metastatic brain tumors, Alzheimer's disease, Huntington's disease, Parkinson's disease, Amyotrophic Lateral Sclerosis (ALS), frontotemporal dementia, hemorrhagic stroke, aneurismal subarachnoid hemorrhage, focal epilepsy, etc.

In one embodiment the biopsy method comprises:
inserting the miniature device of the present invention into the patient via an entry point;
remotely controlling the motion of the miniature device towards the biopsy site;
removing the biopsy sample from the biopsy site for storage in the at least one chamber; and
retrieving the miniature device via an exit point.

In one embodiment the inserting is carried out by any of the following selected from: needle, catheter, or a combination thereof. The remote controlling of the miniature device is at least partially carried out by the system configured to remove and store a biopsy sample utilizing a magnetic inducing apparatus. In one embodiment the motion is carried out in a linear and/or non-linear trajectory through the patient. Thus, the miniature device can be directed along any 3D trajectory/passage through the patient, as required. In one embodiment the miniature device passes through any of the following selected from: tissue, channels, tracts, ducts, cavities, epidural space, vessels, organs, potential space, interfascial planes, interorgan space, or a combination thereof. Channels, passages, tracts, and the likes, can be those that are naturally occurring with the patient, those that are artificially created, or a combination thereof. In one embodiment the channels are pre-made channels, naturally occurring channels, or a combination thereof.

In one embodiment the window is open when at the biopsy site and close when not at the biopsy site. As stated herein, one aspect of the present invention is to transport the biopsy sample from the biopsy site to a location outside of the patient, for inspection by a medical team. Sealing the miniature device at the biopsy site, with the biopsy sample therein, enables the biopsy sample to remain uncontaminated and also prevents the contamination of the biopsy sample to other locations in the body where the miniature device passes. In one embodiment the removing is carried out by moving the miniature device relative to the biopsy site, moving the surgical element relative to the biopsy site, or a combination thereof, whilst is contact with the biopsy site, thereby removing the biopsy sample. In one embodiment the moving of the miniature device relative to the biopsy site, moving of the surgical element relative to the biopsy site, or a combination thereof, is selected from: forward, backward, sideward, up, down, vibration, or a combination thereof.

In one embodiment the method further comprises closing the window of the miniature device, thereby enclosing the biopsy sample in the at least one chamber. In one embodiment the enclosing forms a hermetic seal. As understood herein, the closing of the window; the sealing of the window; and the enclosing of any biopsy sample within a chamber comprised with the miniature device, are all understood interchangeably.

In one embodiment the entry point and the exit point are the same.

In one embodiment the miniature device further comprises a protective coating. In one embodiment the protective coating comprises an antiviral, antibacterial, antifungal component, or a combination thereof.

In one embodiment, the term “a” or “one” or “an” refers to at least one. In one embodiment the phrase “two or more” may be of any denomination, which will suit a particular purpose. In one embodiment, “about” or “approximately” may comprise a deviance from the indicated term of +1%, or in some embodiments, −1%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

Those skilled in the art to which this invention pertains will readily appreciate that numerous changes, variations, and modifications can be made without departing from the scope of the presently disclosed subject matter, mutatis mutandis.

1. A miniature device for use in a system configured to store a biopsy sample collected at a biopsy site in a patient, the miniature device comprising:
at least one steering portion comprising a magnetic material; and
a window comprised in the at least one steering portion through which the biopsy sample passes into at least one chamber within the miniature device.

2. The miniature device of claim 1 further comprising a surgical element comprising at least one sharp edge and configured to remove the biopsy sample for storage within the miniature device.

3. The miniature device of claim 2 wherein the at least one sharp edge is selected from:
smooth edge, serrated edge, hollow grind, beveled edge, and scalloped edge.

4. The miniature device of claim 3 wherein the surgical element is a cylinder comprising a longitudinal opening with the at least one sharp edge, the surgical element being configured to rotate within the miniature device along its longitudinal axis to cut the biopsy sample as the at least one sharp edge passes across the window.

5. The miniature device of claim 4 further configured to seal the at least one chamber when the longitudinal opening is positioned away from the window.

6. The miniature device of claim 3 wherein the surgical element is a cylinder comprising the at least one sharp edge, the surgical element being configured to move backwards to open the window and forwards to close the window along the longitudinal axis of the miniature device, wherein the sharp edge cuts the biopsy sample for storage into the at least one chamber as the sharp edge moves forwards to close the window.

7. The miniature device of claim 3 wherein the surgical element is a cylinder comprising the at least one sharp edge, the surgical element being configured to move backwards to extend out of the window at the back end of the miniature device and forwards to retract into the miniature device along the longitudinal axis of the miniature device, wherein the at least one sharp edge cuts the biopsy sample when extending out of the back end of the miniature device for storage into the at least one chamber when the surgical element retracts into the miniature device.

8. The miniature device of claim 3 wherein the surgical element comprises the at least one sharp edge configured at least partially at the edges of the window at the back end of the miniature device and wherein the biopsy sample is cut when the miniature device moves backwards into the biopsy site.

9. The miniature device of claim 1 wherein the window further comprises a hinged door comprising at least one sharp edge, the at least one sharp edge being positioned such that the biopsy sample is cut and directed into the at least one chamber for storage when the miniature device moves forwards.

10. The miniature device of claim 9 wherein the hinged door is configured to close, sealing the biopsy sample inside the at least one chamber.

11. The miniature device of claim 3 further comprising a pocket door positioned at the side of the window wherein the surgical element is configured to retract into the body of the miniature device via the pocket door to open the window and extend out of the body of the miniature device via the pocket door to close the window thereby cutting the biopsy sample for storage within the at least one chamber.

12. The miniature device of claim 1 further comprising a tapered end.

13. The miniature device of claim 1 wherein the steering portion comprises a non-magnetic shell at least partially surrounding the magnetic material.

14. A system configured to remove and store a biopsy sample at a biopsy site in a patient, the system comprising at least one miniature device according to any one of the preceding claims, the system further comprising a magnetic inducing apparatus configured to be operated to generate a varying magnetic field, thereby remotely controlling motion of the miniature device.

15. A biopsy method comprising:
inserting the miniature device of claim 14 into the patient via an entry point;
remotely controlling the motion of the miniature device towards the biopsy site;
removing the biopsy sample from the biopsy site for storage in the at least one chamber; and
retrieving the miniature device via an exit point.

16. The biopsy method of claim 15 wherein the inserting is carried out by any of the following selected from: needle, catheter, or a combination thereof.

17. The biopsy method of claim 15 wherein the motion is carried out in a linear and/or non-linear trajectory through the patient.

18. The biopsy method of claim 15 wherein the miniature device passes through any of the following selected from: tissue, channels, tracts, ducts, cavities, epidural space, vessels, organs, potential space, interfascial planes, interorgan space, or a combination thereof.

19. The biopsy method of claim 18 wherein the channels are pre-made channels, naturally occurring channels, or a combination thereof.

20. The biopsy method of claim 15 wherein the window is open when at the biopsy site and closed when not at the biopsy site.

21. The biopsy method of claim 15 wherein the removing is carried out by moving the miniature device relative to the biopsy site, moving the surgical element relative to the biopsy site, or a combination thereof, whilst is contact with the biopsy site, thereby removing the biopsy sample.

22. The biopsy method of claim 21 wherein the moving the miniature device relative to the biopsy site, moving of the surgical element relative to the biopsy site, or a combination thereof, is selected from: forward, backward, sideward, up, down, vibration, or a combination thereof.

23. The biopsy method of claim 15 further comprising closing the window of the miniature device, thereby enclosing the biopsy sample in the at least one chamber.

24. The biopsy method of claim 23 wherein the enclosing forms a hermetic seal.

25. The biopsy method of claim 15 wherein the entry point and the exit point are the same.
